(19) United States
(12) Patent Application Publication
(10) Pub. No.: US 2026/0224402 A1
(43) Pub. Date: Aug. 6, 2026

(54) SYSTEMS AND METHODS FOR CONTROLLING LASER TRANSMISSION DURING OPHTHALMIC PROCEDURES

(71) Applicant: Alcon Inc., Fribourg (CH)

(72) Inventor: Kevin Canzone, Coto de Caza, CA (US)

(21) Appl. No.: 19/449,685

(22) Filed: Jan. 15, 2026

Related U.S. Application Data
(60) Provisional application No. 63/754,715, filed on Feb. 6, 2025.

(51) Int. Cl. A61F 9/008

(52) U.S. Cl. CPC A61F 9/008; A61F 2009/00844

(57) ABSTRACT

Embodiments disclosed herein provide a system for controlling laser transmission during an ophthalmic procedure. The system includes first and second laser sources, an optical fiber, and a signal detector. The first and second laser sources are configured to respectively generate first and second laser beams. The optical fiber is configured to receive the first and second laser beams from the first and second laser sources, transmit the first and second laser beams from a distal end of the optical fiber, receive a reflected portion of the second laser beam, and direct the reflected portion to the signal detector. The signal detector is configured to receive the reflected portion from the optical fiber, and generate a signal detector output based on the reflected portion. The signal detector output is indicative of a presence or an absence of a bubble or vapor at the distal end of the optical fiber.

### INTRODUCTION

During ophthalmic surgeries, optical fibers are used to deliver laser beams to a treatment area within an eye. Thermal energy produced by the laser beams can be used to vaporize, cut, emulsify, or cauterize target tissue during, for example, glaucoma surgeries, vitreoretinal procedures, cataract surgeries, etc. To generate the thermal energy, laser systems typically emit continuous or pulsed laser beams at the treatment area via an optical fiber. This thermal energy interacts with surrounding fluids at the treatment area and forms vapor bubbles, which facilitates tissue removal. However, excessive thermal energy may cause tissue to burn or harden (e.g., in a “burning event”), which can limit the effectiveness of the laser beam and increase risks for unintentional damage to surrounding tissues.

Typically, to prevent burning events, surgeons visually detect when tissues are hardening and respond by manually pausing laser transmission and/or adjusting settings of the transmitted laser so that the treatment area does not overheat. However, such burning events can occur rapidly, may be subtle, and/or visually obscured, making them difficult for the surgeon to detect in real time. Monitoring for these burning events can also divert the surgeon’s attention away from other tasks.

### BRIEF SUMMARY

The present disclosure relates generally to systems and methods for controlling laser transmission during ophthalmic procedures.

In certain embodiments, a system for controlling laser transmission during an ophthalmic procedure is provided. The system includes a first laser source, a second laser source, an optical fiber, and a signal detector. The first laser source is configured to generate a first laser beam and the second laser source is configured to generate a second laser beam. The optical fiber is configured to receive the first laser beam from the first laser source and the second laser beam from the second laser source, transmit the first laser beam and the second laser beam from a distal end of the optical fiber, receive a reflected portion of the second laser beam that is reflected back into the distal end of the optical fiber, and direct the reflected portion of the second laser beam to a signal detector. The signal detector is configured to receive the reflected portion of the second laser beam from the optical fiber, and generate a signal detector output based on the reflected portion of the second laser beam, the signal detector output indicative of a presence or an absence of a bubble or vapor at the distal end of the optical fiber.

In certain embodiments, a method for controlling laser transmission during an ophthalmic procedure is provided. The method includes generating, by a first laser source, a first laser beam, and generating, by a second laser source, a second laser beam. The method further includes receiving, by an optical fiber, the first laser beam from the first laser source and the second laser beam from the second laser source, transmitting, by the optical fiber, the first laser beam and the second laser beam at a treatment area, receiving, at the optical fiber, a reflected portion of the second laser beam from an interface between the treatment area and the optical fiber, and directing, by the optical fiber, the reflected portion of the second laser beam to a signal detector. The method further includes receiving, at the signal detector, the reflected portion of the second laser beam from the optical fiber, and generating, by the signal detector, a signal detector output based on the reflected portion of the second laser beam, the signal detector output indicative of a presence or an absence of a bubble or vapor at a distal end of the optical fiber.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 shows an example system for controlling laser transmission during an ophthalmic procedure, according to certain embodiments.

FIG. 2A shows a flowchart of a method for operating the system of FIG. 1, according to certain embodiments.

FIG. 2B shows a flowchart of various blocks for controlling the laser transmission of the system of FIG. 1, according to certain embodiments.

FIG. 3 shows another example system for controlling laser transmission during an ophthalmic procedure, according to certain embodiments.

FIG. 4A shows a flowchart of another method for operating the system of FIG. 3, according to certain embodiments.

FIG. 4B shows a flowchart of various blocks for controlling the laser transmission of the system of FIG. 3, according to certain embodiments.

FIG. 5 shows a schematic diagram of an example controller included in the systems of FIGS. 1 and 3, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

Reference throughout this specification to the term “distal” refers to a system, device, component, end, portion, or segment that is disposed closer to a patient and/or further from a console during an ophthalmic procedure; and the term “proximal” refers to the system, device, component, end, portion, or segment that is disposed further from the patient and/or closer to the console during the ophthalmic procedure.

Current laser systems often emit the laser beam(s) via optical fibers continuously, or in a series of pulses. The continuous or short, high-energy laser pulses generate thermal energy at the treatment area to vaporize and/or remove tissue. The thermal energy heats surrounding fluids (e.g., aqueous humor, vitreous humor, interstitial or intracellular fluids, blood, water, balanced salt solution (BSS), or the like), causing vapor and bubbles (e.g., vapor bubbles) to form at an emitting end (distal end or optical fiber tip) of the optical fiber. The laser pulses also generate pressure waves in the liquid medium, which further break and emulsify tissue.

However, in some instances, when too much thermal energy or heat is produced at the treatment area, for example, to the point where the tissue starts to burn or harden, the hardened tissue may inhibit the transmission of the laser beam by partially or completely blocking the emitting end of the optical fiber. As an example, the tissue may burn or harden due to excessive transmission of the laser beam, a lack of fluid surrounding the emitting end of the optical fiber, and/or the emitting end of the optical fiber being positioned too close to the treatment area. Such burning of the tissue may be referred to herein as a “burning event.” Burning events are harmful because when tissue hardens, it becomes less responsive to further treatment and generally reduces laser effectiveness. Excessive heat can also damage surrounding tissues and/or cause scarring, which can lead to other optical complications. Further, burnt tissue may form an occlusion at the emitting end of the optical fiber, thereby obstructing laser transmission and complicating ophthalmic procedures by requiring the surgeon to clean or replace the optical fiber tip.

To avoid burning events, surgeons often have to visually recognize that a burning event is occurring (or about to occur) and then manually alter the transmission of the laser beam, interrupting the procedure. For example, when the surgeon observes a burning event (or potential burning event), the surgeon typically stops transmission of the laser beam to allow fluids at the treatment area to flow back into the treatment area and around the emitting end of the optical fiber. In some instances, the surgeon may also have to change certain settings (e.g., power, duration, and/or wavelength of the laser beam) to avoid overheating tissue at the treatment area.

However, having the surgeon monitor for burning events may divert the surgeon’s focus away from other aspects of the procedure (e.g., laser positioning, tissue response, etc.) and/or increase fatigue throughout the procedure. The surgeon may also not always recognize when a burning event is occurring because it can occur rapidly, subtly, and/or may be obscured by fluids, lighting conditions, or a limited field of view.

Accordingly, the embodiments described herein provide laser systems and methods for controlling laser transmission. For example, laser systems as described herein use back-reflected light, sound waves, and/or pressure changes to detect whether vapor is present at an emitting end of an optical fiber, which thereby allows the laser system to recognize whether the optical fiber is occluded. By recognizing whether the optical fiber is occluded, the laser system is able to control transmission of the laser beam to mitigate burning events and avoid unintentional trauma to other ocular tissues. The laser systems and methods described herein, therefore facilitate safer, quicker, and more efficient ophthalmic procedures.

Further, during ophthalmic procedures, the surgeon may prematurely transmit a laser beam before the optical fiber is near the treatment area (i.e., the optical fiber is outside the eye), or accidentally remove the optical fiber from the eye while transmitting the laser beam. In both instances, the laser beam may be unintentionally directed at tissue outside the eye, causing unwanted damage thereto.

To avoid such accidental transmission of the laser beam, the laser systems and methods herein recognize whether the optical fiber is disposed near the treatment area based on whether vapor is present or absent at the emitting end of the optical fiber. Thus, by determining whether the optical fiber is near the treatment area, the laser system can prohibit transmission of the laser beam, thereby preventing accidental transmission of laser beams and damage to tissues outside the eye.

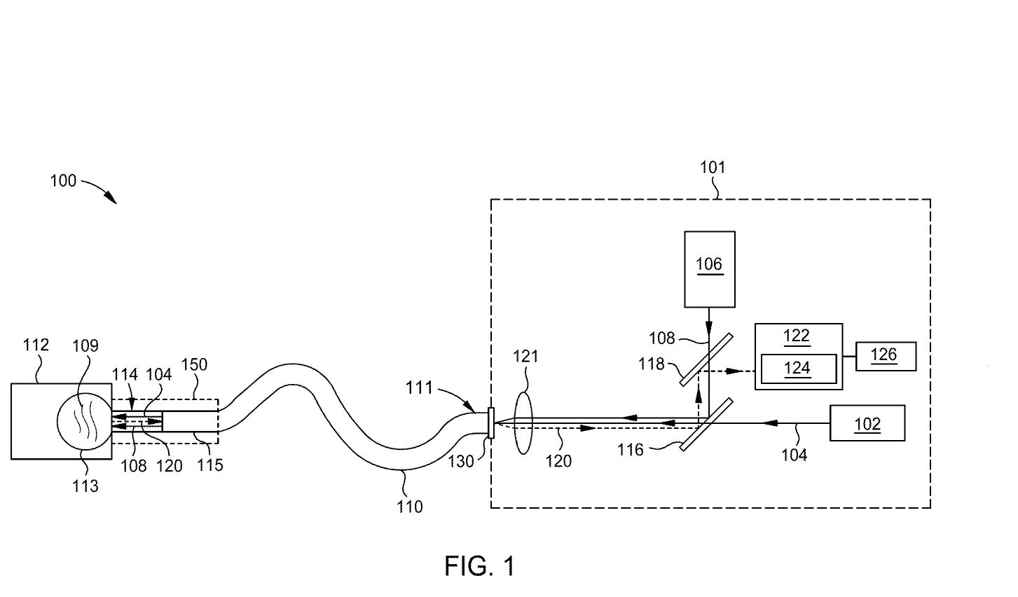

FIG. 1 shows an example system 100 for controlling laser transmission during an ophthalmic procedure, according to certain embodiments. The system 100 includes a surgical laser system 101, which may be operably coupled to and/or in communication with a surgical console, such as a surgical console for ophthalmic surgical procedures. The surgical laser system 101 comprises a first laser source 102 configured to generate a first laser beam 104, and a second laser source 106 configured to generate a second laser beam 108.

In some embodiments, the first laser source 102 is a treatment laser source configured to generate the first laser beam 104 for treatment of a patient. The first laser beam 104 may be used for cutting and/or emulsifying material during a surgical operation. For example, the first laser beam 104 may be used as a treatment beam for performing various functions during ophthalmic surgical procedures (e.g., vitreoretinal procedures, glaucoma surgeries, cataract surgeries, etc.).

In some embodiments, the first laser beam 104 generated by the first laser source 102 is an ultraviolet (“UV”) (<350 nanometers (nm)) laser light. In some embodiments, the first laser beam 104 is an infrared (“IR”) (780 – 4000 nm) laser light, such as a mid-IR laser light. In some embodiments, the first laser beam 104 is an argon blue-green laser light (488 nm), a Nd-YAG (neodymium-doped yttrium aluminum garnet) laser light (532 nm) such as a frequency-doubled Nd-YAG laser light, a krypton red laser light (647 nm), or any other suitable type of laser light for ophthalmic surgery. In some embodiments, the first laser beam 104 has a wavelength of about 500 nm.

In some embodiments, the first laser source 102 may generate and propagate the first laser beam 104 having a pulse rate within a range of about 100 hertz (Hz) and 10 kilohertz (kHz). In some embodiments, the first laser source 102 may generate and propagate the first laser beam 104 having a pulse rate within a range of about 10 kHz and about 500 kHz, or between about 1 kHz and about 1500 Hz. Other pulse rate ranges are contemplated as well. In some examples, the first laser source 102 produces a nanosecond, a picosecond, or a femtosecond first laser beam 104. In some embodiments, the first laser source 102 is a continuous wave (CW) laser source that can be switched to a pulsed mode.

The surgical laser system 101 also includes the second laser source 106 configured to generate the second laser beam 108. In some embodiments, the second laser beam 108 may be configured to operate as a source of illumination of a surgical site, for aiming, or the like. In some embodiments, the second laser beam 108 generated by the second laser source 106 is a visible (380 – 780 nm) or IR (780 – 1000 nm) laser light or illumination beam. For example, in certain embodiments, the second laser beam 108 comprises a 640 nm laser light. However, other spectrums/ranges (e.g., 400 nm to 4 μm) are further contemplated for the second laser beam 108.

In some embodiments, the second laser source 106 may generate and transmit the second laser beam 108 having a pulse rate within a range of about 100 Hz and 10 kHz. In some embodiments, the second laser source 106 may generate and transmit the second laser beam 108 having a pulse rate within a range of about 10 kHz and about 5 megahertz (MHz), or between about 1 kHz and about 1500 Hz. Other pulse rate ranges are contemplated as well. In some examples, the second laser source 106 produces a nanosecond, a picosecond, or a femtosecond second laser beam 108. In some embodiments, the second laser source 106 may produce a continuous coherent or semi-continuous second laser beam 108. For example, the second laser source 106 may produce a continuous wave second laser beam 108 at low power.

The system 100 further includes an optical fiber 110 having a proximal end 111 configured to be removably coupled to a port 130 of the surgical laser system 101. The optical fiber 110 is configured to proximally receive and distally propagate both of the first laser beam 104 and the second laser beam 108 generated by the first laser source 102 and the second laser source 106, respectively, which may be disposed adjacent to the proximal end 111 of the optical fiber 110.

The optical fiber 110 may include any suitable type of optical fiber configured to transmit light energy along a length of the optical fiber 110. In some embodiments, the optical fiber 110 may, at least in part, be made of germanium oxide-based glass, sapphire, fluoride, zirconium fluoride, and/or silica. The optical fiber 110 may include a single material, a blend of materials, may have different regions of different materials, etc. However, any suitable materials or space for the efficient propagation of laser beams 104 and 108 are contemplated.

The optical fiber 110 may be at least partially clad, single-clad, double clad, multi-clad, or may be unclad. In embodiments with cladding, the cladding may be concentric with one or more cores of the optical fiber 110. In some embodiments, the first laser beam 104 and/or the second laser beam 108 may be propagated through the optical fiber 110 via a cladding.

In some embodiments, the optical fiber 110 has a single core structure. In such embodiments, the first laser beam 104 and the second laser beam 108 may be propagated along the same core of the optical fiber 110. In other embodiments, the optical fiber 110 has a multi-core structure. In such embodiments, the first laser beam 104 and the second laser beam 108 may be propagated along the same core or different cores of the optical fiber 110.

Generally, the optical fiber 110 may be rigid or flexible. In some embodiments, the optical fiber 110 may be straight or tapered. In some embodiments, a diameter of the optical fiber 110 is between about 100 μm (micrometers) and about 400 μm (e.g., between 100 μm and 300 μm, 100 μm and 200 μm, 200 μm and 400 μm, 200 μm and 300 μm, or 300 μm and 400 μm). In some embodiments, the optical fiber 110 may have different regions having similar or different geometries to one another. In such embodiments, the different regions may comprise one or more pieces of optical fiber butt-coupled to each other.

In FIG. 1, the optical fiber 110 is partially disposed within, and/or is integrated with, a surgical handpiece 150, which is shown by a dashed box for clarity purposes. The surgical handpiece 150 may be any suitable ophthalmic surgical instrument that can be operated on the basis of the embodiments described herein. For example, the surgical handpiece 150 may be a laser probe (also referred to as a “surgical laser probe” or a “laser device”).

The optical fiber 110 is also configured to distally receive and proximally propagate a reflected portion 120 of the second laser beam 108 that is reflected back by, and into, a distal end 115 of the optical fiber 110 during ophthalmic procedures. The reflected portion 120 may be passed through the optical fiber 110 along the same core or a different core of the optical fiber 110 as at least one of the first laser beam 104 or the second laser beam 108 being propagated through the optical fiber 110 in the opposite direction.

The optical fiber 110 includes an optical fiber tip 114 disposed at the distal end 115 of the optical fiber 110 opposite the first laser source 102 and/or the second laser source 106. Generally, the first laser beam 104 and the second laser beam 108 may be transmitted (i.e., emitted, directed, output, etc.) distally from the optical fiber tip 114 after being propagated through the optical fiber 110. The optical fiber tip 114 may be fabricated of similar or different construction from another portion of the optical fiber 110. For example, the optical fiber tip 114 may vary from another portion of the optical fiber 110 in material, material properties, optical properties, geometry, or the like. For example, the optical fiber tip 114 may be rigid while another portion of the optical fiber 110 may include a flexible portion to allow for positioning of the optical fiber tip 114 relative to a treatment area 112 or a surgical site (e.g., within an eye).

In some embodiments, the optical fiber 110 and/or the system 100 may further include one or more optical elements configured to direct, re-direct, filter, polarize, focus, collimate, split, or otherwise manipulate the first laser beam 104, the second laser beam 108, and/or the reflected portion 120 of the second laser beam 108. For example, in FIG. 1, a first dichroic mirror 116 and a second dichroic mirror 118 are depicted. Generally, the dichroic mirrors 116 and 118 may facilitate either the reflection or transmission of laser beams depending on their wavelengths. In FIG. 1, the first dichroic mirror 116 is depicted as facilitating transmission of the first laser beam 104 and re-direction (e.g., reflection) of the second laser beam 108 into the optical fiber 110, while also facilitating re-direction of the proximally-travelling reflected portion 120 of the second laser beam 108 toward the second dichroic mirror 118. The second dichroic mirror 118 then re-directs the reflected portion 120 into a signal detector 122 (e.g., an optical signal detector or any suitable type of signal detector).

In the illustrated embodiment of FIG. 1, a lens 121 is also shown. The lens 121 may be configured to focus at least one of the first laser beam 104 or the second laser beam 108 onto the optical fiber 110 at or through the port 130. For example, the lens 121 may be configured to focus at least one of the first laser beam 104 or the second laser beam 108 onto a core of the optical fiber 110.

The system 100 further includes the signal detector 122, which is configured to receive the reflected portion 120 and generate a signal detector output based on the reflected portion 120. In some embodiments, the optical fiber 110 is configured to direct the reflected portion 120 of the second laser beam 108 to the signal detector 122 indirectly (e.g., via an air gap or other gap). In some other embodiments, the optical fiber 110 may direct the second laser beam 108 to the signal detector 122 through direct contact transmission.

The signal detector 122 includes a sensor 124 (e.g., photodiode, optical sensor, or other energy sensitive detector element) capable of detecting the reflected portion 120 of the second laser beam 108 incident at the signal detector 122. In certain embodiments, the sensor 124 generates a sensor signal based on the detected reflected portion 120 of the second laser beam 108, which the signal detector 122 then uses to generate a signal detector output. The signal detector output may be communicated as a signal intensity voltage (V) measurement that corresponds to an index of refraction, where the change in the index of refraction indicates a change of Fresnel coefficients at the optical fiber tip 114. In other words, the signal detector output is indicative of transient changes of the reflected portion 120, the transient changes being caused by changes of a refractive index at the optical fiber tip 114 during water-vapor and vapor-water transitions.

In some embodiments, the signal detector output may be electrically amplified. In some other embodiments, the signal detector output may pass through a high-pass filter to separate the transient back-reflection signal from a direct current (DC) baseline, or the high-pass filter may be used prior to or after the amplifier, or between amplifier stages.

The signal detector 122 is coupled to a controller 126 that is configured to receive and analyze the signal detector output from the signal detector 122, and determine various metrics/characteristics of the reflected portion 120. Examples of the metrics/characteristics may include a duration of a transient signal, pulse width, amplitude, frequency, etc. Such metrics/characteristics of the reflected portion 120 are utilized to determine a presence or an absence of vapor 109 (e.g., within a bubble 113) at the optical fiber tip 114, which is generated by at least the first laser source 102 at the optical fiber tip 114, as described in further detail below. As an example, the signal detector output may correspond to a refractive index, where changes in the refractive index indicate the presence or absence of the bubble 113 formed by the vapor 109. A duration of the signal detector output may also correspond to a lifetime of the bubble 113.

The system 100 may be used, for example, to treat tissue at the treatment area 112 (e.g., within a patient’s eye). As such, the optical fiber 110 is configured to direct (or emit) the first laser beam 104 and the second laser beam 108 at the treatment area 112. In some embodiments, during use, the optical fiber 110 may be positioned to be at least partially disposed near the treatment area 112, or positioned to contact a surface of the treatment area 112.

When the optical fiber 110 emits the first laser beam 104 distally towards the treatment area 112, thermal energy of the first laser beam 104 heats the tissue and/or surrounding fluid (e.g., aqueous humor, vitreous humor, interstitial or intracellular fluids, blood, water, BSS, or the like) at the treatment area 112. The tissue and/or surrounding fluid at the treatment area 112 absorbs the thermal energy of the first laser beam 104 and undergoes a phase change (e.g., vaporization) into vapor 109, which causes a bubble 113 (e.g., vapor bubble) to form at the optical fiber tip 114. The bubble 113 may also contain air, argon, or other gas instead of, or in combination with, the vapor 109. In certain embodiments, each subsequent emission/firing of the first laser beam 104 from the first laser source 102 may generate vapor 109. Continued transmission of the first laser beam 104 at the treatment area 112 may cause the vapor 109 to expand within the bubble 113 and thereby increase in volume before ultimately collapsing. When the bubble 113 collapses, it separates from the optical fiber tip 114. During operation, bubbles repeatedly form and collapse at the optical fiber tip 114.

Simultaneously with the first laser beam 104, the second laser beam 108 is generated and propagated distally through the optical fiber 110 and out of the optical fiber tip 114. The second laser beam 108 is thus emitted to an interface between the treatment area 112 and the optical fiber tip 114. As an example, the interface includes the vapor 109 within the bubble 113 and/or an inner surface of the bubble 113.

In response to the presence or absence of vapor 109 (and the bubble 113) at the optical fiber tip 114, for example, due to the thermal energy of first laser beam 104, the second laser beam 108 is subject to a change in index of refraction at the interface between the treatment area 112 and the optical fiber tip 114. For example, the index of refraction before formation of the vapor 109 and the bubble 113 may be approximately equal to 1.33 between the optical fiber tip 114 and the treatment area 112. Once the bubble 113 is formed by the vapor 109, the index of refraction may change to approximately 1.0 at the interface between the optical fiber tip 114 and the vapor 109 within the bubble 113.

The change in the index of refraction at the optical fiber tip 114 results in a change of the Fresnel coefficients of the optical fiber tip 114 and, thus, the optical behavior of the second laser beam 108 at the optical fiber tip 114, whereby an increased amount of the second laser beam 108 is back-reflected through the optical fiber 110. For example, with the vapor 109 present, a larger portion of the second laser beam 108 may be transiently reflected by the optical fiber tip 114 proximally through the optical fiber 110 while a smaller portion of the second laser beam 108 may pass into the treatment area 112. In some embodiments, a portion of the transmitted part of the second laser beam 108 may be further reflected from the inner surface of the bubble 113 back into the optical fiber tip 114.

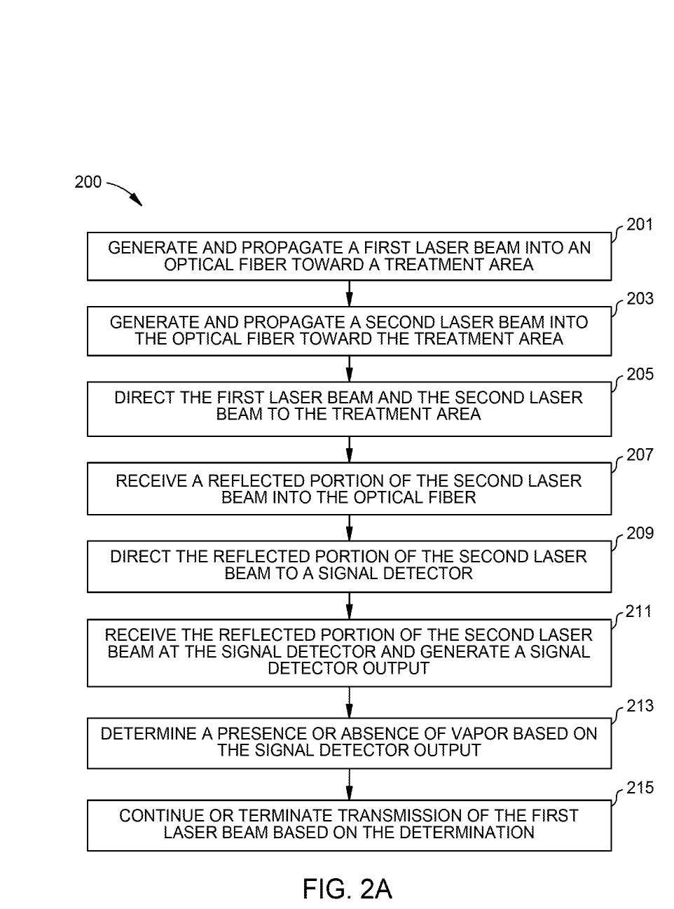

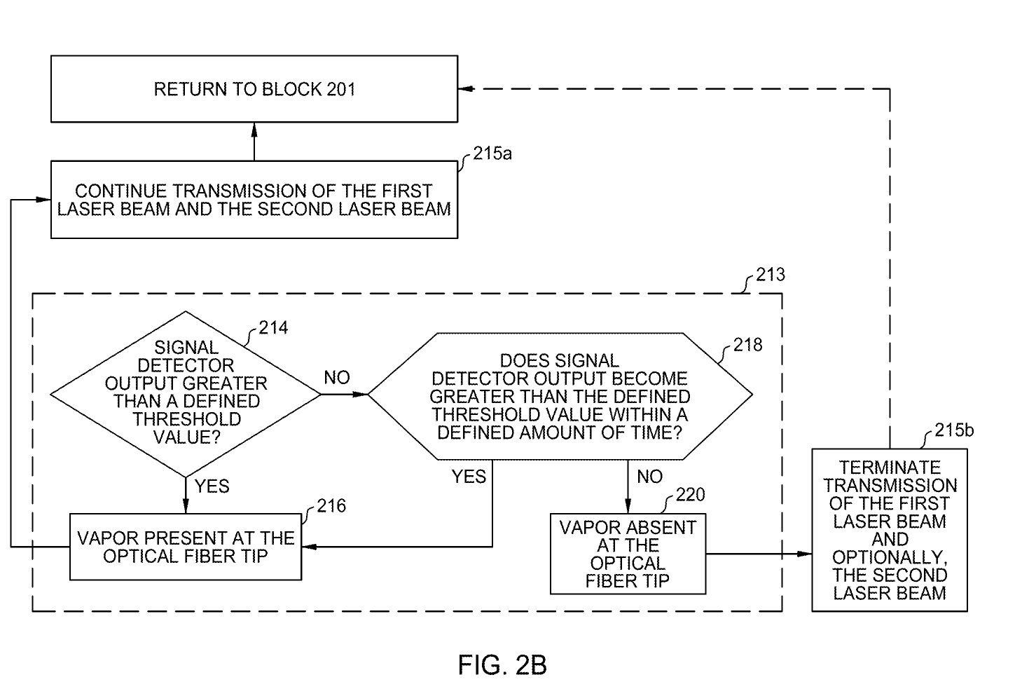

The change in optical behavior of the second laser beam 108 is detected by the sensor 124 of signal detector 122, which continuously or non-continuously monitors/detects for the reflected portion 120 of the second laser beam 108. The signal detector 122 then sends the signal detector output to the controller 126 based on a sensor signal of the sensor 124. The controller 126 coupled therewith analyzes the signal detector output to determine metrics/characteristics of the reflected portion 120, which can be correlated with the presence or absence of the vapor 109 at the optical fiber tip 114. Methods for determining whether the vapor 109 is present at the optical fiber tip 114, and for operating the system 100 are described with respect to FIGS. 2A-2B.

Please note that although the signal detector 122 and controller 126 are shown as integrated components of the surgical laser system 101 in FIG. 1, the signal detector 122 and controller 126 may be separate components operably coupled with the surgical laser system 101.

In some embodiments, the first laser beam 104 and the second laser beam 108 may be generated by a single laser source of the surgical laser system 101 that is configured to produce two or more types of laser beams, or laser beams having different characteristics. For example, in some embodiments, the first laser beam 104 and the second laser beam 108 may be generated by the first laser source 102. In some other embodiments, the first laser beam 104 and the second laser beam 108 may be generated by the second laser source 106.

In certain embodiments, additional optical components and/or relays are also contemplated for use with the system 100. For example, the system 100 can include additional optical components in the form of wave plates (e.g., half wave plates or quarter wave plates), polarizing elements (e.g., polarizers or polarizing cubes), reflectors, dichroic elements, lenses (e.g., focusing lenses, collimators, etc.), and the like. In some examples, the optical components may be used to facilitate power modification, laser light isolation, transmission of identified wavelengths, etc. One or more components may include coatings (e.g., anti-reflective coating), materials, gratings, films, etc. to separate wavelengths, isolate the laser beams (e.g., laser beams 104, 108) from the back-reflected beams (e.g., reflected portion 120) or the like. In some embodiments, physical structures may be used to transmit light. In other embodiments, light may be transmitted through free space. In some embodiments, light may be transmitted via a combination of physical structures and free space.

FIG. 2A shows a flowchart of a method 200 for operating the system 100 of FIG. 1, according to certain embodiments.

At block 201, the first laser source 102 generates and propagates the first laser beam 104 into the optical fiber 110 and toward the treatment area 112.

At block 203, the second laser source 106 or the first laser source 102 generates and propagates the second laser beam 108 into the optical fiber 110 and toward the treatment area 112. In some embodiments, the second laser beam 108 is generated simultaneously with the first laser beam 104. In some embodiments, the second laser beam 108 is generated sequentially relative to the first laser beam 104. For example, the second laser beam 108 may be generated prior to generating the first laser beam 104, or vice versa.

In certain embodiments, the first laser beam 104 and the second laser beam 108 are automatically generated at blocks 201 and 203, respectively, in response to the user pressing and releasing an “ON” button, in some embodiments, or in response to the user holding an “ON” button, in some other embodiments.

At block 205, the optical fiber 110 directs (e.g., transmits) the first laser beam 104 and the second laser beam 108 at the treatment area 112. The first laser beam 104 reacts with surrounding fluids (e.g., aqueous humor, vitreous humor, interstitial or intracellular fluids, blood, water, BSS, or the like) at the treatment area 112, which forms the vapor 109 and the bubble 113 at the optical fiber tip 114. The formation of the vapor 109 and the bubble 113 changes the refractive index of the treatment area 112 and therefore, the reflection coefficient of an interface (i.e., the vapor 109 and/or the inner surface of the bubble 113) between the optical fiber tip 114 and the treatment area 112.

At block 207, the optical fiber 110 receives a portion (e.g., reflected portion 120) of the second laser beam 108 that is back-reflected from the interface between the optical fiber tip 114 and the treatment area 112. Some of the second laser beam 108 may also reflect from within the bubble 113 and return into the optical fiber 110.

At block 209, the optical fiber 110 directs the reflected portion 120 of the second laser beam 108 to the signal detector 122 (such as by using one or more optics or relays).

At block 211, the sensor 124 receives the reflected portion 120 of the second laser beam 108 and generates a sensor signal based on the received reflected portion 120. The signal detector 122 then generates a signal detector output based on the sensor signal generated by the sensor 124 upon receiving the reflected portion 120 of the second laser beam 108. The signal detector 122 then communicates (or relays) the signal detector output to the controller 126.

In some embodiments, the signal detector output may be electrically amplified. In some other embodiments, the signal detector output may pass through a high-pass filter to separate the transient back-reflection signal from the DC baseline, or the high-pass filter may be used in front or after the amplifier, or between amplifier stages.

At block 213, the controller 126 determines the presence or absence of the vapor 109 at the optical fiber tip 114 based on the signal detector output. Determining whether the vapor 109 is present or absent at the optical fiber tip 114 is described in further detail with respect to FIG. 2B.

Note that the presence or absence of the vapor 109 is indicative of the presence, absence, and/or lifetime of the bubble 113 because the bubble 113 is formed by the vapor 109. In other words, determining the presence or absence of the vapor 109 includes determining the presence, absence, and/or lifetime of the bubble 113. As such, even though the steps of the method 200 refer to the detection of the presence or absence of the vapor 109, the detection of the presence or absence of the vapor 109 includes a determination of, or is synonymous with, the detection of the presence or absence of the bubble 113.

At block 215, the controller 126 determines to either continue or terminate the transmission of the first laser beam 104 and optionally the second laser beam 108 based on the determination at block 213.

FIG. 2B shows a flowchart of various blocks illustrating how the determination of the presence or absence of the vapor is made at block 213. The flowchart of FIG. 2B also illustrates how laser transmission of the system 100 of FIG. 1 is controlled at block 215 based on the determination performed at block 213, according to certain embodiments.

As shown in FIG. 2A, at block 213, the controller 126 determines the presence or absence of the vapor 109 at the optical fiber tip 114 based on the signal detector output. To determine whether the vapor 109 is present or absent at the optical fiber tip 114, at block 214, the controller 126 determines whether the signal detector output, which corresponds to the signal intensity voltage, is greater than a defined threshold value by comparing the signal detector output to the defined threshold value. Whether the signal detector output is greater than the defined threshold value is indicative of the presence or absence of vapor 109 at the optical fiber tip 114.

The signal detector output generally rises as the presence of vapor 109 increases, for example, due to the first laser beam 104 and the second laser beam 108 being properly transmitted at the treatment area 112, and the optical fiber tip 114 increasingly receiving the reflected portion 120. In other words, the signal detector output generally rises as a difference between the refractive indices of the optical fiber 110 and the treatment area 112 increases. On the other hand, the signal detector output may subsequently fall as the presence of vapor 109 decreases, for example, due to the transmission of the first laser beam 104 being terminated or an occlusion at the optical fiber tip 114, and the optical fiber tip 114 decreasingly receiving the reflected portion 120. In other words, the signal detector output generally falls as the difference between the refractive indices of the optical fiber 110 and the treatment area 112 decreases. The rise and fall of the signal detector output therefore corresponds to the formation and collapse of the bubble 113. As such, the signal detector output being greater than the defined threshold value indicates the presence of vapor 109 at the optical fiber tip 114.

As described below in more detail, by determining the presence or absence of vapor 109 at the optical fiber tip 114, the controller 126 can determine whether the laser beams 104, 108 are being properly transmitted at the optical fiber tip 114, whether the optical fiber tip 114 is occluded, and/or whether the optical fiber tip 114 is disposed near the treatment area 112 (e.g., is not disposed outside the eye).

If the signal detector output is greater than the defined threshold value (“YES”), then at block 216, the controller 126 determines that vapor 109 is present at the optical fiber tip 114. Because vapor 109 is present at the optical fiber tip 114, the optical fiber tip 114 is not occluded and the laser beams 104, 108 are being properly transmitted at the treatment area 112. Accordingly, the controller 126 determines that the laser beams 104, 108 can continue to be transmitted at the treatment area 112 at block 215a. Thus, the method 200 returns to block 201 of method 200 in FIG. 2A.

If the signal detector output is not greater than (i.e., is less than or equal to) the defined threshold value (“NO”), then at block 218, the controller 126 continues to monitor the signal detector output to determine whether the signal detector output becomes greater than the defined threshold value within a defined amount of time (e.g., within 1 second, 2 seconds, 3 seconds, 4 seconds, or 5 seconds). For example, during an ophthalmic procedure, the signal detector output may temporarily be less than or equal to the defined threshold value after the bubble 113 collapses (or pops) and before a new bubble is formed at the optical fiber tip 114.

If the signal detector output becomes greater than the defined threshold value within the defined amount of time (“YES”), then at block 216, the controller 126 determines that vapor 109 is present (i.e., reformed) at the optical fiber tip 114. Accordingly, the controller 126 determines that the optical fiber tip 114 is not occluded and the laser beams 104, 108 can continue to be transmitted at the treatment area 112 at block 215a. The method 200 then returns to block 201 of method 200 in FIG. 2A.

If the signal detector output does not rise above the defined threshold value within the defined amount of time (“NO”), then at block 220, the controller 126 determines that vapor 109 is absent at the optical fiber tip 114. The signal detector output may not rise above the defined threshold value within the defined amount of time, for example, due to partial or complete occlusion of the optical fiber tip 114, and/or the optical fiber tip 114 not being disposed near the treatment area 112 (e.g., optical fiber tip 114 is not disposed inside the eye).

For example, the optical fiber tip 114 may become partially or completely occluded due to tissue fragments, coagulated proteins, and/or cellular debris building up at the optical fiber tip 114. For example, due to excessive and/or continuous thermal energy from the first laser beam 104, such materials may gradually clump together and stick to the optical fiber tip 114, thereby obstructing the propagation of the first laser beam 104, the second laser beam 108, and/or the reflected portion 120. Consequently, when the optical fiber tip 114 is occluded, the occlusion can prevent effective delivery of laser energy to the treatment area 112. The surgeon may then remove the optical fiber tip 114 from the treatment area 112 to clean or replace the optical fiber tip 114 to maintain adequate transmission of the first laser beam 104 and the second laser beam 108. In such an example, signal detector output does not rise above the defined threshold value within the defined amount of time.

In yet another example, signal detector output not rising above the defined threshold value within the defined amount of time may also indicate that the optical fiber tip 114 is not disposed near the treatment area 112 (e.g., optical fiber tip 114 is not disposed within the eye). For example, in some cases, the surgeon may prematurely activate transmission of the laser beams 104, 108 before the optical fiber tip 114 is inserted into the eye, or accidentally remove the optical fiber tip 114 from the eye during the procedure. Accordingly, the optical fiber tip 114 does not receive the reflected portion 120 because the second laser beam 108 is not back-reflected, which results in the signal detector output being less than or equal to the defined threshold value for longer than the defined amount of time.

In the examples above, once the controller 126 determines that vapor 109 is absent at the optical fiber tip 114, at block 215b, the controller 126 terminates transmission of the first laser beam 104 and optionally, the second laser beam 108 to avoid unintentionally burning tissue near the treatment area 112 or tissue outside the eye. Note that in some embodiments, the transmission of the first laser beam 104 is terminated while the second laser beam 108 continues to be transmitted because the second laser beam 108 is an aiming laser beam that does not burn tissue. However, in some embodiments, the controller 126 terminates transmission of both the first laser beam 104 and the second laser beam 108.

In certain embodiments, transmission of the first laser beam 104 and optionally the second laser beam 108 is automatically terminated. As an example, the first laser beam 104 and optionally, the second laser beam 108 may be terminated for a defined termination period that is greater than 1 millisecond and less than 1 second. As another example, the first laser beam 104 and optionally, the second laser beam 108 may be terminated for a defined termination period that is greater than 1 second (e.g., greater than 2 seconds, 3 seconds, 4 seconds, or 5 seconds). By terminating transmission of the first laser beam 104, water and/or other fluid at the treatment area 112 can flow back in front of the optical fiber tip 114, such that when the first laser beam 104 is later retransmitted, the first laser beam 104 will interact with the fluid, instead of emulsifying tissue at the treatment area 112.

Once the transmission of the first laser beam 104 and optionally, the second laser beam 108 are terminated for the defined termination period, the method 200 returns to block 201 of method 200 in FIG. 2A. As such, the first laser beam 104 and the second laser beam 108 are retransmitted by the surgical system 100. For example, the first laser beam 104 and the second laser beam 108 are automatically retransmitted, or can be manually reactivated by the surgeon after the defined termination period expires. In other words, transmission of the first laser beam 104 and the second laser beam 108 is resumed. In certain embodiments, once the termination period expires, the surgeon is notified via an audible sound, visual indicator, or other form of notification from the surgical handpiece 150 or the system 100.

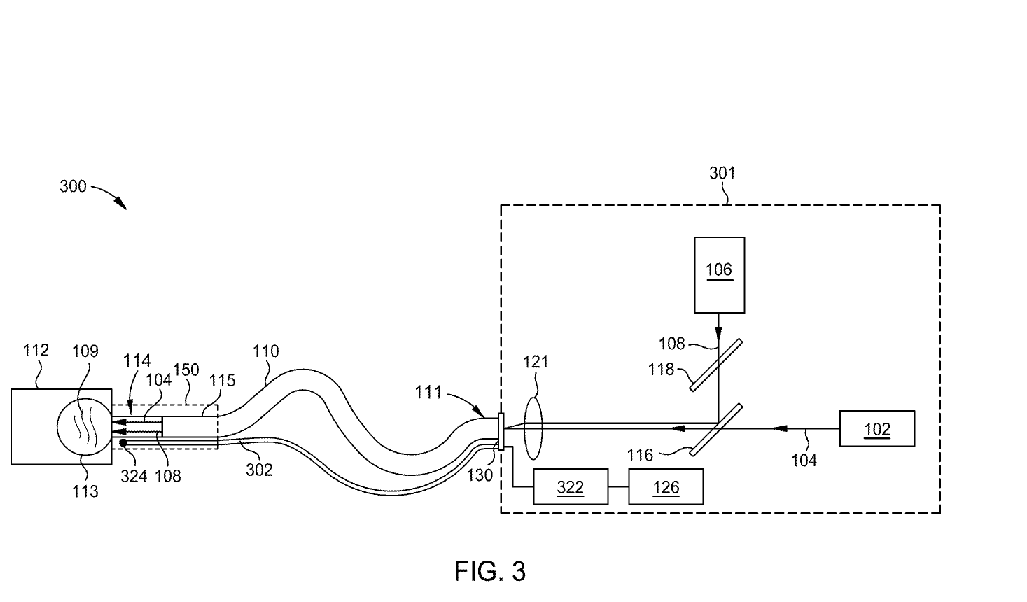

FIG. 3 shows another example system 300 for controlling laser transmission during an ophthalmic procedure, according to certain embodiments. The system 300 operates and includes components similar to the system 100 shown in FIG. 1. However, as shown in FIG. 3, a sensor 324 of the system 300 is disposed at the optical fiber tip 114, and is coupled to a signal detector 322 of a surgical laser system 301 via a cable 302. In certain embodiments, the cable 302 may be disposed alongside the optical fiber 110 and proximally coupled to the surgical laser system 301 via the port 130.

In FIG. 3, the sensor 324 may be a microphone, a pressure sensor, and/or a similar sensor configured to detect the presence or absence of the vapor 109 (and the bubble 113) at the optical fiber tip 114. For example, the sensor 324 is configured to generate a sensor signal that is indicative of whether the vapor 109 (and bubble 113) is present or absent at the optical fiber tip 114. The sensor signal is directed to the signal detector 322 via the cable 302. Note that, although in the embodiments of FIG. 3 the optical fiber 110 and the cable 302 are shown separately, in some other embodiments, they can both be disposed in a single sleeve or tubing.

In embodiments where the sensor 324 is a microphone, the sensor 324 is configured to generate the sensor signal based on sound (or audio) at the optical fiber tip 114. For example, when the first laser beam 104 causes vapor 109 and the bubble 113 to form at the optical fiber tip 114, the bubble 113 may rapidly expand and collapse, resulting in a “popping” or “clicking” sound having an acoustic signal in the form of an audible or ultrasonic sound wave. The expansion and collapse of the bubble 113 may be measured using a frequency range or amplitude in decibels (dB). Repetitive “popping” or “clicking” sounds are indicative of the presence of vapor 109, and characteristic of proper transmission of the first laser beam 104 because bubbles rapidly expand and collapse during certain ophthalmic procedures involving laser energy. Alternatively, if repetitive “popping” or “clicking” sounds are not detected, then the optical fiber tip 114 is occluded or not disposed near the treatment area 112 (e.g., is not disposed within the eye).

In embodiments where the sensor 324 is a pressure sensor, the sensor 324 is configured to generate a sensor signal based on pressure at the optical fiber tip 114. For example, when the first laser beam 104 causes vapor 109 and the bubble 113 to form at the optical fiber tip 114, the bubble 113 may rapidly expand and collapse, resulting in pressure changes at the optical fiber tip 114. Repetitive increases and/or decreases in pressure are indicative of the presence of vapor 109, and characteristic of proper transmission of the first laser beam 104 because bubbles rapidly expand and collapse during certain ophthalmic procedures involving laser energy. Alternatively, if repetitive pressure increases are not detected (or pressure is continuously increasing), then the optical fiber tip 114 is occluded or not disposed near the treatment area 112 (e.g., is not disposed within the eye).

Based on the sensor signal received from the sensor 324, signal detector 322 then generates a signal detector output, as described with reference to FIG. 1. As an example, the signal detector output may be communicated as a signal intensity voltage (V) measurement that corresponds to sound waves or pressure changes detected by the sensor 324 at the optical fiber tip 114.

By using the microphone, the pressure sensor, and/or a similar sensor, the system 300 is able to detect the presence of vapor 109 without having to rely on the reflected portion (e.g., reflected portion 120) of the second laser beam 108 to detect the presence of vapor 109.

Note that a surgical laser system may utilize any combination of the signal detectors and sensors described herein. For example, in some embodiments, a surgical laser system may utilize both a microphone and a pressure sensor to detect the presence of vapor 109 with even more accuracy. In some other embodiments, the surgical laser system may use a microphone, pressure sensor, and/or an optical sensor (e.g., sensor 124) for further accuracy.

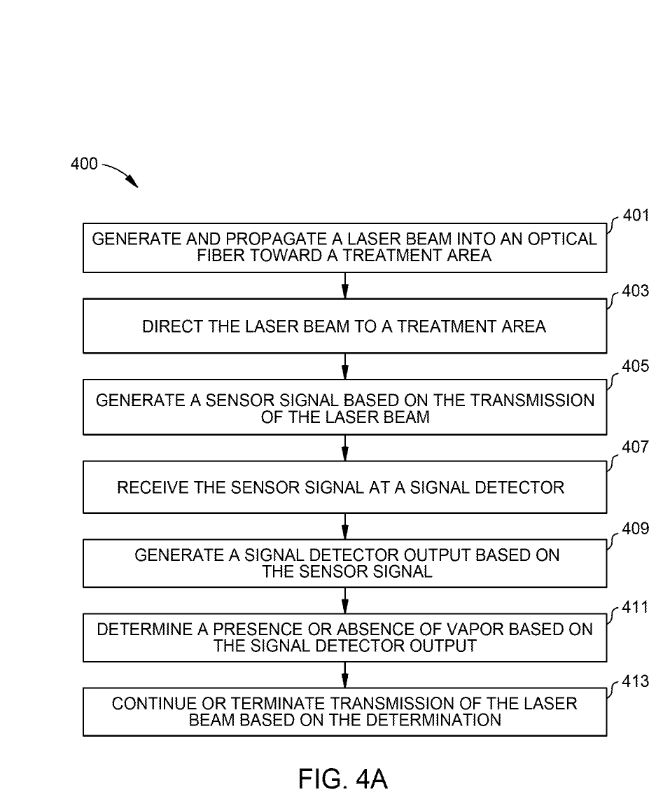

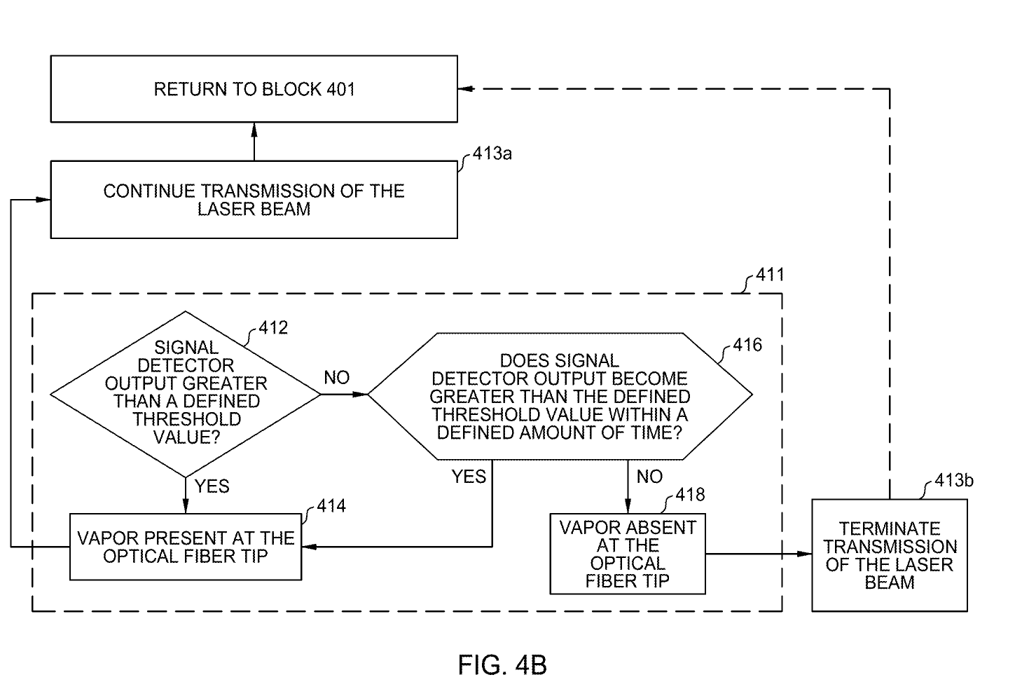

FIG. 4A shows a flowchart of a method 400 for operating the system 300 of FIG. 3, according to certain embodiments.

At block 401, a laser source (e.g., first laser source 102) generates and propagates a laser beam (e.g., first laser beam 104) into the optical fiber 110 and toward the treatment area 112. In certain embodiments, the first laser beam 104 is automatically generated in response to the user pressing and releasing an “ON” button, in some embodiments, or in response to the user holding an “ON” button, in some other embodiments. Note that the method 400 may also involve the second laser source 106 generating and propagating the second laser beam 108 as described with respect to block 203 of FIG. 2A.

At block 403, the optical fiber 110 directs (e.g., transmits) the first laser beam 104 at the treatment area 112. The first laser beam 104 reacts with surrounding fluids (e.g., aqueous humor, vitreous humor, interstitial or intracellular fluids, blood, water, BSS, or the like) at the treatment area 112, which repeatedly forms vapor and bubbles at the optical fiber tip 114. The repeated formation of the vapor 109 and the bubble 113 produces corresponding sound waves and pressure changes at the optical fiber tip 114 as described with respect to FIG. 3.

At block 405, the sensor 324 (e.g., microphone or pressure sensor) of the system 300 generates a sensor signal subsequent to the transmission of the first laser beam 104 at the treatment area 112. For example, the sensor 324 generates the sensor signal based on detected sound waves or pressure changes caused by transmitting the first laser beam 104 at the treatment area 112. The sensor signal is then directed to the signal detector 322 via the cable 302.

At block 407, the signal detector 322 receives the sensor signal from the sensor 324 via the cable 302.

At block 409, the signal detector 322 generates a signal detector output based on the sensor signal generated by the sensor 324. As an example, the signal detector output corresponds to a signal intensity voltage. The signal detector 322 then communicates (or relays) the signal detector output to the controller 126.

At block 411, the controller 126 determines the presence or absence of vapor 109 at the optical fiber tip 114 based on the signal detector output. Determining whether the vapor 109 is present or absent at the optical fiber tip 114 is described in further detail with respect to FIG. 4B.

Note that the presence or absence of the vapor 109 is indicative of the presence, absence, and/or lifetime of the bubble 113 because the bubble 113 is formed by the vapor 109. In other words, determining the presence or absence of the vapor 109 includes determining the presence, absence, and/or lifetime of the bubble 113.

At block 413, the controller 126 determines whether to continue or terminate transmission of the first laser beam 104 based on the determination at block 411. The controller 126 may also determine whether to continue or terminate transmission of the second laser beam 108 as described with respect to block 215 of FIG. 2A.

FIG. 4B shows a flowchart of various blocks illustrating how the determination of the presence or absence of the vapor is made at block 411. The flowchart of FIG. 4B also illustrates how laser transmission of the system 300 of FIG. 3 is controlled at block 413 based on the determination performed at block 411, according to certain embodiments.

As shown in FIG. 4A, at block 411, the controller 126 determines the presence or absence of the vapor 109 at the optical fiber tip 114 based on the signal detector output. To determine whether the vapor 109 is present or absent at the optical fiber tip 114, at block 412, the controller 126 determines whether the signal detector output, which corresponds to the signal intensity voltage, is greater than a defined threshold value by comparing the signal detector output to the defined threshold value. Whether the signal detector output is greater than the defined threshold value is indicative of the presence or absence of vapor 109 at the optical fiber tip 114. As described below in more detail, by determining the presence or absence of vapor 109 at the optical fiber tip 114, the controller 126 can determine whether the first laser beam 104 is being properly transmitted at the optical fiber tip 114, whether the optical fiber tip 114 is occluded, and/or whether the optical fiber tip 114 is disposed near the treatment area 112 (e.g., is not disposed outside the eye).

If the signal detector output is greater than the defined threshold value (“YES”), then at block 414, the controller 126 determines that vapor 109 is present at the optical fiber tip 114. Because vapor 109 is present at the optical fiber tip 114, the optical fiber tip 114 is not occluded and the first laser beam 104 is being properly transmitted at the treatment area 112. Accordingly, the controller 126 determines that the first laser beam 104 can continue to be transmitted at the treatment area 112 at block 413a. Thus, the method 400 returns to block 401 of the method 400 in FIG. 4A.

If the signal detector output is not greater than (i.e., is less than or equal to) the defined threshold value (“NO”), then at block 416, the controller 126 continues to monitor the signal detector output to determine whether the signal detector output becomes greater than the defined threshold value within a defined amount of time (e.g., within 1 second, 2 seconds, 3 seconds, 4 seconds, or 5 seconds). For example, during an ophthalmic procedure, the signal detector output may temporarily be less than or equal to the defined threshold value after the bubble 113 collapses (or pops) and before a new bubble is formed at the optical fiber tip 114.

If the signal detector output becomes greater than the defined threshold value within the defined amount of time (“YES”), then at block 414, the controller 126 determines that vapor 109 is present (i.e., reformed) at the optical fiber tip 114. Accordingly, the controller 126 determines that the optical fiber tip 114 is not occluded and the first laser beam 104 can continue to be transmitted at the treatment area 112 at block 413a. Thus, the method 400 returns to block 401 of the method 400 in FIG. 4A.

If the signal detector output does not rise above the defined threshold value within the defined amount of time (“NO”), then at block 418, the controller 126 determines that vapor 109 is absent from the optical fiber tip 114. The signal detector output may not rise above the defined threshold value within the defined amount of time, for example, due to partial or complete occlusion of the optical fiber tip 114, and/or the optical fiber tip 114 not being disposed near the treatment area 112 (e.g., optical fiber tip 114 is not disposed inside the eye).

In the examples above, once the controller 126 determines that vapor 109 is absent at the optical fiber tip 114, at block 413b, the controller 126 terminates transmission of the first laser beam 104. Note that as described above with respect to block 215b of FIG. 2B, in some embodiments, the transmission of the first laser beam 104 is terminated while the second laser beam 108 continues to be transmitted because the second laser beam 108 is an aiming laser beam that does not burn tissue. However, in some embodiments, the controller 126 terminates transmission of both the first laser beam 104 and the second laser beam 108.

In certain embodiments, transmission of the first laser beam 104 is automatically terminated. As an example, the first laser beam 104 may be terminated for a defined termination period that is greater than 1 millisecond and less than 1 second. As another example, the first laser beam 104 may be terminated for a defined termination period that is greater than 1 second (e.g., greater than 2 seconds, 3 seconds, 4 seconds, or 5 seconds). By terminating transmission of the first laser beam 104, water and/or other fluid at the treatment area 112 can flow back in front of the optical fiber tip 114, such that when the first laser beam 104 is later retransmitted, it will interact with the fluid, instead of emulsifying tissue at the treatment area 112.

Once the transmission of the first laser beam 104 is terminated for the defined termination period, the method 400 returns to block 401 of method 400 in FIG. 4A. As such, the first laser beam 104 is retransmitted by the surgical system 300. For example, the first laser beam 104 is automatically retransmitted, or can be manually reactivated by the surgeon after the defined termination period expires. In other words, transmission of the first laser beam 104 is resumed. In certain embodiments, once the termination period expires, the surgeon is notified via an audible sound, visual indicator, or other form of notification from the surgical handpiece 150 or the system 300.

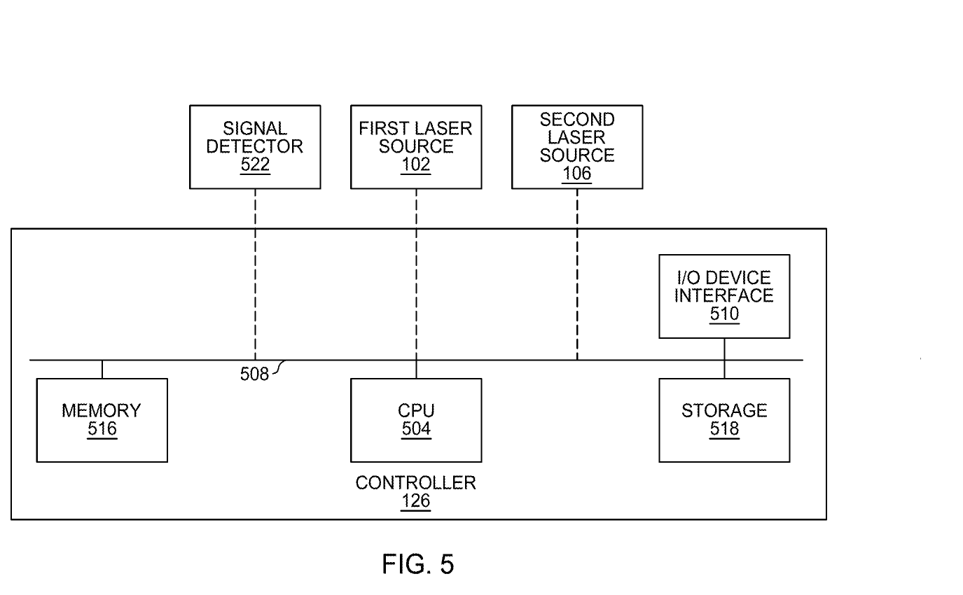

FIG. 5 shows a schematic diagram of an example controller, such as the controller 126 of the systems 100 and 300 of FIGS. 1 and 3, respectively, according to certain embodiments. In some embodiments, the controller 126 may be integrated with or operably coupled with a surgical console. The controller 126 includes at least one I/O (Input/Output) device interface 510, which may allow for the connection of various I/O devices (e.g., footswitches, keyboards, displays, mouse devices, pen input, voice, etc.) to the controller 126. The controller 126 is in wired or wireless communication with the first laser source 102, the second laser source 106, and the signal detector 522 (which represents the signal detector 122 of FIG. 1, the signal detector 322 of FIG. 3, or a combination of both), via the interconnect 508. In some embodiments, in addition to or separately from the controller 126, the first laser source 102, the second laser source 106, and the signal detector 522 may be integrated with or operably coupled with a surgical console.

The controller 126 further includes a Central Processing Unit (CPU) 504, a memory 516, and a storage 518. The CPU 504 is configured to retrieve and execute programming instructions stored in the memory 516. Similarly, the CPU 504 may retrieve and store application data residing in the memory 516. The interconnect 508 transmits programming instructions and application data, among the CPU 504, I/O device interface 510, memory 516, storage 518, first laser source 102, second laser source 106, and the signal detector 522, etc. The CPU 504 may include a single CPU, multiple CPUs, a single CPU having multiple processing cores, and the like. The memory 516 may be random access memory (RAM), and the storage 518 may be a disk drive. Moreover, the memory 516 and/or storage 518 may be any type of a readily available memory, such as RAM, read only memory (ROM), floppy disk, hard disk, solid state, flash memory, magnetic memory, or any other form of digital storage, local or remote. In certain embodiments, the memory 516 and/or storage 518 include instructions, which when executed by the CPU 504, can affect determinations/measurements of laser transmission by the first laser source 102 and/or the second laser source 106 based on data received from the signal detector 522. In certain embodiments, the CPU 504, memory 516, and storage 518 may be the main processor and memory of controller 126.

In the embodiment of FIG. 5, the CPU 504 of the controller 126 may include an integrated circuit capable of performing logic functions. In this manner, the CPU 504 is in the form of a standard integrated circuit package with power, input, and output pins. In other embodiments, the CPU 504 is a microprocessor. In other cases, the CPU 504 is not a programmable microprocessor, but instead is a special purpose controller.

In the embodiment of FIG. 5, the controller 126 receives signals from the signal detector 522. These signals, for example, may include signal detector outputs corresponding to reflected light (e.g., reflected portion 120), sound waves, and/or pressure changes received at the sensor 124 and/or 324.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

### Example Embodiments

Embodiment 1: A system for controlling laser transmission during an ophthalmic procedure, the system comprising: a laser source configured to generate a laser beam; an optical fiber configured to: receive the laser beam from the laser source; and transmit the laser beam from a distal end of the optical fiber; a signal detector, configured to: receive a sensor signal based on the transmission of the laser beam; and generate a signal detector output based on the sensor signal, the signal detector output indicative of a presence or an absence of a bubble or vapor at the distal end of the optical fiber.

Embodiment 2: The system of Embodiment 1, wherein system further comprises: a controller, configured to: take one or more actions based on the signal detector output.

Embodiment 3: The system of Embodiment 2, wherein the one or more actions comprise determining the bubble or the vapor is present when the signal detector output is greater than a defined threshold value.

Embodiment 4: The system of Embodiment 2, wherein the one or more actions comprise determining the bubble or the vapor is absent when the signal detector output is less than or equal to a defined threshold value.

Embodiment 5: The system of Embodiment 1, wherein the system further comprises: a sensor disposed at the distal end of the optical fiber and configured to direct the sensor signal to the signal detector.

Embodiment 6: The system of Embodiment 5, wherein the sensor is a microphone.

Embodiment 7: The system of Embodiment 5, wherein the sensor is a pressure sensor.

What is claimed is:

1. A system for controlling laser transmission during an ophthalmic procedure, the system comprising:
a first laser source configured to generate a first laser beam;
a second laser source configured to generate a second laser beam;
an optical fiber configured to:
receive the first laser beam from the first laser source and the second laser beam from the second laser source;
transmit the first laser beam and the second laser beam from a distal end of the optical fiber;
receive a reflected portion of the second laser beam that is reflected back into the distal end of the optical fiber; and
direct the reflected portion of the second laser beam to a signal detector;
the signal detector, configured to:
receive the reflected portion of the second laser beam from the optical fiber; and
generate a signal detector output based on the reflected portion of the second laser beam, the signal detector output indicative of a presence or an absence of a bubble or vapor at the distal end of the optical fiber.

2. The system of claim 1, wherein the system further comprises:
a controller configured to:
take one or more actions based on the signal detector output.

3. The system of claim 2, wherein the one or more actions comprise determining the bubble or the vapor is present when the signal detector output is greater than a defined threshold value.

4. The system of claim 2, wherein the one or more actions comprise determining the bubble or the vapor is absent when the signal detector output is less than or equal to a defined threshold value.

5. The system of claim 4, wherein the one or more actions comprise automatically terminating the output of the first laser beam when the signal detector output is less than or equal to the defined threshold value for longer than a defined amount of time.

6. The system of claim 5, wherein the controller is further configured to:
resume transmission of the first laser beam after a defined termination period expires.

7. The system of claim 1, wherein the signal detector output corresponds to a signal intensity voltage that is based on the reflected portion of the second laser beam.

8. The system of claim 1, wherein the presence or the absence of the bubble or the vapor indicates whether there is an occlusion at the distal end of the optical fiber.

9. The system of claim 1, wherein:
the first laser beam comprises a treatment beam, and
the second laser beam comprises an illumination beam.

10. The system of claim 9, wherein:
the treatment beam is an infrared laser light, and
the illumination beam is a visible laser light.

11. A method for controlling laser transmission during an ophthalmic procedure, the method comprising:
generating, by a first laser source, a first laser beam;
generating, by a second laser source, a second laser beam;
receiving, by an optical fiber, the first laser beam from the first laser source and the second laser beam from the second laser source;
transmitting, by the optical fiber, the first laser beam and the second laser beam at a treatment area;
receiving, at the optical fiber, a reflected portion of the second laser beam from an interface between the treatment area and the optical fiber;
directing, by the optical fiber, the reflected portion of the second laser beam to a signal detector;
receiving, at the signal detector, the reflected portion of the second laser beam from the optical fiber; and
generating, by the signal detector, a signal detector output based on the reflected portion of the second laser beam, the signal detector output indicative of a presence or an absence of a bubble or vapor at a distal end of the optical fiber.

12. The method of claim 11, further comprising:
taking, by a controller, one or more actions based on the signal detector output.

13. The method of claim 12, wherein the one or more actions comprise determining the bubble or the vapor is present when the signal detector output is greater than a defined threshold value.

14. The method of claim 12, wherein the one or more actions comprise determining the bubble or the vapor is absent when the signal detector output is less than or equal to a defined threshold value.

15. The method of claim 14, wherein the one or more actions comprise automatically terminating the output of the first laser beam when the signal detector output is less than or equal to the defined threshold value for longer than a defined amount of time.
